(19) United States
(12) Patent Application Publication
(10) Pub. No.: US 2011/0301717 A1
(43) Pub. Date: Dec. 8, 2011

(54) Supporting and Forming Transitional Material for Use in Supporting Prosthesis Devices, Implants and to Provide Structure in a Human Body

(76) Inventor: Hilton Becker, Boca Raton, FL (US)

(21) Appl. No.: 12/832,161

(22) Filed: Jul. 8, 2010

Related U.S. Application Data
(60) Provisional application No. 61/351,062, filed on Jun. 3, 2010.

(51) Int. Cl. A61F 2/02

(52) U.S. Cl. 623/23.72

(57) ABSTRACT

A transitional mesh and thread for use in the human body is dissolved. The transitional mesh being comprised of sections of non-absorbable mesh fabric with sections of absorbable mesh fabric, such that each non-absorbable section is attached to absorbable mesh fabric. The non-absorbable mesh sections can be overlaid with absorbable mesh which after it is absorbed leaves the non-absorbable mesh in an array without connection to each other. The thread to be used to create the mesh has non-absorbable fibers that can be discontinuous, loosely woven or embedded in an absorbable material. The fabric mesh itself can be loosely woven and coated with absorbable material. The patterns of non-absorbable mesh and space between non-absorbable mesh sections can be varied to provide various strengths and degrees of motion and movement. The mesh can also be coated with materials to reduce infection, provide tissue growth, reduce scar tissue or other medicinal purposes.

### FIELD OF THE INVENTION

This invention relates to a forming and supporting material usable for breast reconstruction following a mastectomy, breast augmentation or modification, or the treatment of breast implant complications, especially capsular contraction and more particularly to a method for forming and supporting a breast implant in a human body. The material may also be utilized in other areas of reconstructive surgery where initial non-yielding, strong support is required or desired to be provided which after a suitable period of time, when healing has occurred, transforms into a flexible matrix material having the characteristic of human tissue acting as a scaffold that allows host tissue in-growth without restriction of elasticity, flexibility but flexibility and motion.

### BACKGROUND FOR THE INVENTION

Implants and methods for breast reconstruction and augmentation are well known and have been used for over forty years. The two primary difficulties with implants have been the issue of supporting and restraining the implant to maintain its location and to supply an extra layer of tissue over the implant while allowing appropriate movement and preventing fibrous scar tissue encapsulation. The problems of providing appropriate support while maintaining the desired movement have been dealt with in many of the patent applications discussed herein. The greater support provided the less natural movement is achieved. This problem is amplified by the creation of scar tissue. When a foreign body is implanted into the body, the implant material is walled off by the response of the human tissue. This is commonly referred to as encapsulation. As the capsule that is formed is scar tissue, it is fairly rigid and in certain cases may actually contract, resulting in hardness around the implant. The encapsulation can also lead to the problem of spherical scar contracture. The scar tissue surrounds the prosthetic device and as it contracts causes the configuration of the implant to be altered as well as creating hardness, discomfort, displacement and pain. Implants are traditionally placed under the muscle or partially under the muscle when performing breast reconstruction. This is done to provide an extra layer of tissue over the implant. By providing a material which acts as a scaffold over the implant it would not be necessary to perform additional surgery to elevate the muscle over the implant.

Numerous approaches have been proposed to address these problems. Cronin, U.S. Pat. No. 3,293,663 filed on Dec. 27, 1966, was one of the first implant patents for breast prosthesis that proposed support use of corrugated fabric against the chest wall, which is anchored to the back portion of the implant. The Perras U.S. Pat. No. 3,665,520 filed on May 30, 1972, later proposed support using a Dacron strip affixed to the back wall of the implant. Frank, U.S. Patent Application No. U.S. 2007/0088434A1 filed on Nov. 29, 2006, proposed support using a sheet of prosthetic material configured to form a sling-shaped receiving area to support the breast implant.

Popov PCT Application W02007/004214A2 filed Jun. 28, 2006, uses a basket-shaped structure to provide support to an implant or the mammary gland itself.

These proposals provided support but did not fully allow natural motion, nor did they deal with the problem of encapsulation.

A second approach has been to have the implant surface textured or modified to be bio-compatible to provide support and hopefully reduce capsule formation. This is seen in McGhan, U.S. Pat. No. 6,913,626B2 filed on Jul. 5, 2005, and Agerup, Patent Application. No. 2008/0312739A1 filed on Dec. 18, 2008. There has been only moderately success with such procedures and later studies have indicated it has not achieved the desired objectives.

Another approach has been use of an outer container or pouch to hold the implant prosthesis and interact with the body by in-growth to provide support and try to reduce the occurrence of capsular contracture's inhibiting properties.

The use of sheet or film type materials which are biologically absorbable into the body to provide support has been tried and is disclosed in Naficy, U.S. Pat. No. 4,298,998 filed on Nov. 10, 1981. The use of non-bioabsorbable mesh coated with a bioabsorbable material is seen in Buevich, U.S. Patent App. 2008/0132922A1 filed on Jun. 6, 2008, and using biocompatible but non-bioabsorbable meshes to provide supporting structure is disclosed in Chen, U.S. Patent App. 2009/0082864A1 filed on Mar. 26, 2009, O′Keefe, U.S. Pat. No. 4,936,858 filed on Jun. 26, 1990, and Maxwell, U.S. Patent App. 2009/0125107A1 filed on May 14, 2009. However, the use of these various meshes has not resolved the problem related to breast or other type implants as corrective operation rates to address need for more natural movement and capsular contracture have been unacceptably high.

The use of fabric type materials has been known for some time, the purpose of which has been to provide the function of a permanent re-enforcing structure. These materials have usually incorporated a permanent non-absorbable structure which at times is laced together or coated with various bioabsorbable portions used as filler type material. See Landi, U.S. Pat. No. 5,326,355 filed on Jul. 5, 1994, and Ledergerber, U.S. Pat. No. 4,955,907 filed on Sep. 11, 1990. Typically these fabric type materials contain a weave or structure, usually a knotted mesh, that will be of a biocompatible, non-absorbable material which may have a bio-absorbable component. After absorbance of any bio-absorbable component, the remaining structure which is intended to provide needed support provides insufficient strength and in many cases cause scar tissue to form about the permanent, non-absorbable material resulting in the scar tissue formation, discomfort and hardness, as well as need for corrective surgery. The use of mesh to provide additional support and use of textured surfaces to promote tissue growth and affix the implant is taught in co-pending U.S. patent application Ser. No. 12/169,000 filed on Jul. 8, 2008 which is a continuation in part of U.S. patent application Ser. No. 12/026,032 filed on Feb. 5, 2008. Also incorporated by reference are co-pending applications U.S. patent application Ser. No. 12/552,352 filed on Sep. 2, 2009, U.S. patent application Ser. No. 12/556,050 filed on Sep. 9, 2009, and U.S. patent application Ser. No. 12/026,032 filed on Feb. 5, 2008. However, none of these prior art attempts have solved the problem of providing both adequate support, strength, elasticity and scaffold function with acceptable control or elimination of capsular contracture, scar tissue formation, discomfort and hardness.

The prior art did not achieve support with flexibility and motion. Clinical surgical re-cutting had to be performed or previously utilized fabric support had to be done to achieve flexibility and motion. This invention performs such surgical result without need for surgical intrusion.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a segmented fabric comprising sections which are permanent threads or strands, i.e. non-absorbable and sections which are absorbable, such that the fabric transitions from a continuous sheet to an array of non-absorbable sections which are not connected to each other. As the absorbable portion dissipates, the implant and tissue are provided structural support by the non-absorbable portions of the fabric. The areas between the non-absorbable segments are natural body tissues that can move naturally and elastically. Since the absorbable fabric areas dissipate and provide disruptions to a continuous type of structure, there is much less scar tissue formed and capsular contracture does not become a problem. The material can be conformed into a variety of mesh configurations and reduced to single strands with intermittent absorbable material, can also provide support and be used as a suture or area done by the physician during the surgery.

It is the desire of this invention by virtue of its unique and novel approach to provide the following benefits to breast implant and other restorative procedures; provide support but allow natural movement, prevent or reduce to nominal levels capsular contracture, allow use of smooth surfaced gel implant prosthesis more effectively, with placement above the muscle, stop or minimize rippling from occurring, provide treatment to encourage in-growth and against potential infections, and provide the scaffold for the forming of a new tissue layer that is permanent but elastic, simulating natural human supportive tissue.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### DESCRIPTION OF DRAWINGS

FIG. 1 illustrates a transitional mesh material using a hexagonal non-absorbing area separated by an absorbing area.

FIG. 1(a) illustrates the transitional mesh before utilization.

FIG. 1(b) illustrates the transitional mesh after utilization and absorption has occurred.

FIG. 2 illustrates a transitional mesh where the absorbable mesh portion is overlaid on the sections of a non-absorbable material.

FIG. 3 illustrates the mesh utilized for an implant device, shaped in conical fashion.

FIG. 4 illustrates the mesh utilized for an implant device, shaped in a basket or pocket configuration.

FIG. 5 illustrates a mesh utilizing knotted connections representing non-yielding mesh.

FIG. 6 illustrates a mesh utilizing an unknotted weave and intentional breaks in the weave representing a yielding mesh.

FIG. 7(a) illustrates a loose absorbable weave mesh housing fine yielding woven non-absorbable material.

FIG. 7(b) illustrates the mesh of FIG. 7(a) after absorption has occurred with remaining yielding woven non-absorbable material mesh.

FIG. 7(c) illustrates a thread woven with absorbable material housing fine strands of non-absorbable material.

FIG. 7(d) illustrates the thread weave of FIG. 7(c) after absorption has occurred and shows the intentional break in non-absorbable fibers.

FIG. 8(a) illustrates the thread fibers, non-absorbable material wrapped around the absorbable material.

FIG. 8(b) illustrates the non-absorbable thread fibers after absorption of the absorbable material has occurred.

FIG. 9 illustrates a sponge-type sheet of absorbable material with non-absorbable threads contained therein.

FIG. 10 lists the acts for utilizing the method for repair or support of tissue areas.

FIG. 11 lists the acts for utilizing the method in area of the body for prosthetic devices or implants.

FIG. 12 lists the acts for utilizing the method for providing support by constructing an array of non-absorbing mesh for prosthetic devices or implants.

FIG. 13 lists the acts for utilizing the method for providing support by constructing an array of non-absorbing mesh for repair or support of tissue areas.

### DESCRIPTION OF THE PREFERRED EMBODIMENTS OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention overcomes the disadvantages of the prior art by incorporating a segmented mesh material that provides the needed initial support when a breast implant is being inserted or reconstructive surgery is being performed, yet prevents capsule constriction from occurring by forming a substratum for tissue in-growth which is elastic and allows natural movement.

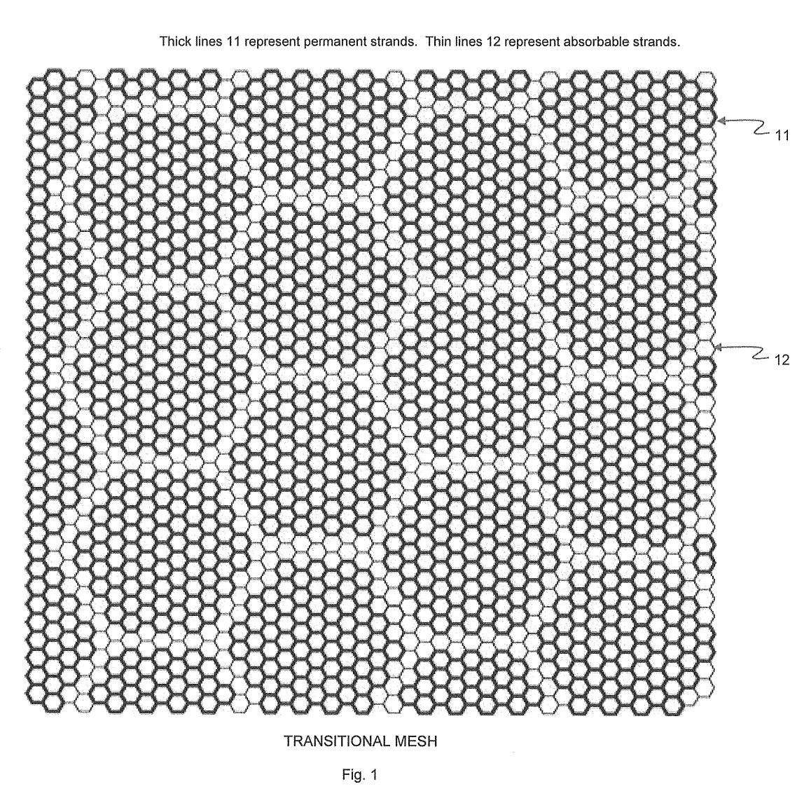

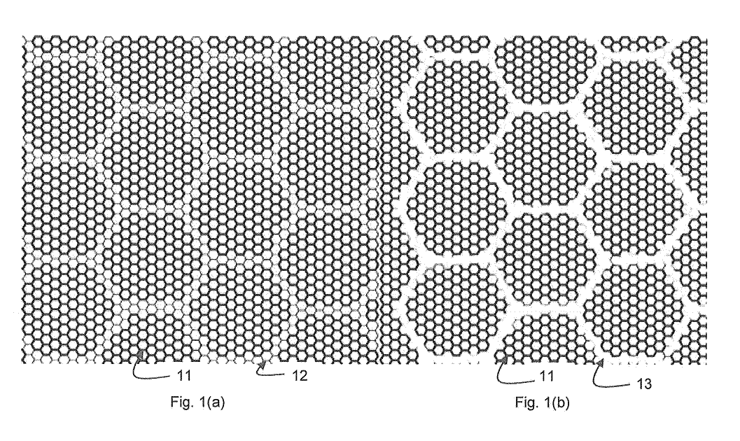

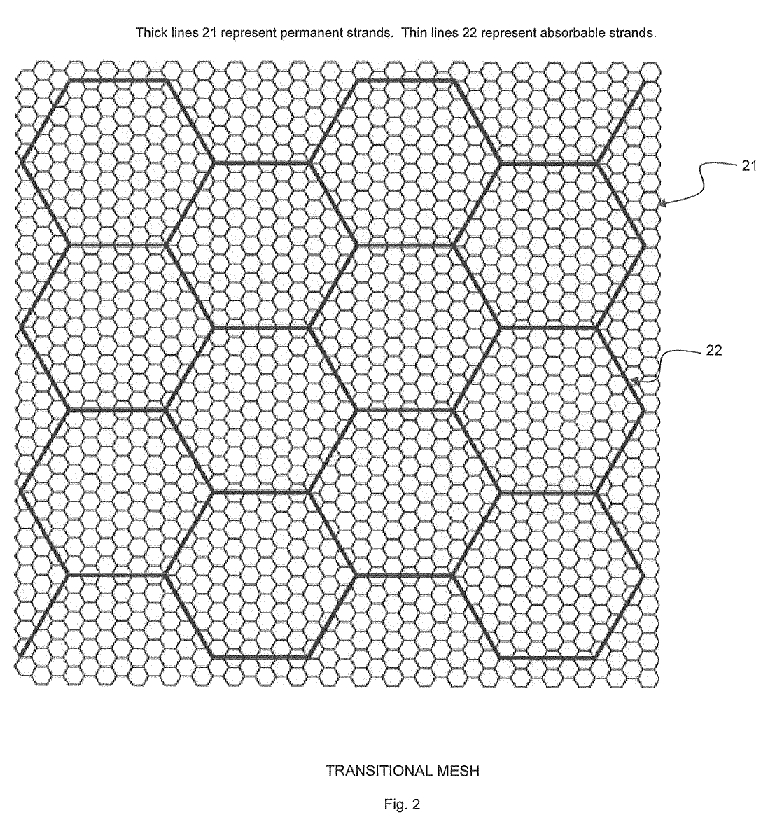

The present invention in a preferred embodiment FIG. 1 shows a segmented fabric comprising sections which are permanent threads or strands, i.e. non-absorbable 11 and sections which are absorbable 12, such that the fabric transitions from a continuous sheet to an array of non-absorbable sections which are not connected to each other. We call this a transitional mesh since the mesh changes its structural composition. The term “transitional” can also apply to a thread which utilizing this invention will also change its structural composition.

The absorbable material 11 can be, but is not required to be, of equal stiffness and strength to that of the non-absorbable material, allowing the mesh fabric to provide support to the implant during the healing process. As the absorbable portion dissipates, the implant and tissue are provided structural support by the non-absorbable portions of the fabric 12. The areas between the non-absorbable segments are natural body tissues that can move naturally and elastically. Since the absorbable fabric areas dissipate and provide disruptions to a continuous type of structure, there is much less scar tissue formed and capsular contracture does not become a problem. FIG. 1(a) shows the transitional mesh material when initially utilized adjacent to a view FIG. 1(b) of the transitional mesh material after absorption takes place. FIG. 1(b) shows the areas 13 where the absorbable portion of the mesh has dissolved.

The transitional mesh fabric shown in FIG. 1 can be fabricated with the absorbable material threaded or woven between the non-absorbable portions, or it can be constructed such that a fabric of absorbable materials is pressed or overlaid upon or between the non-absorbable components.

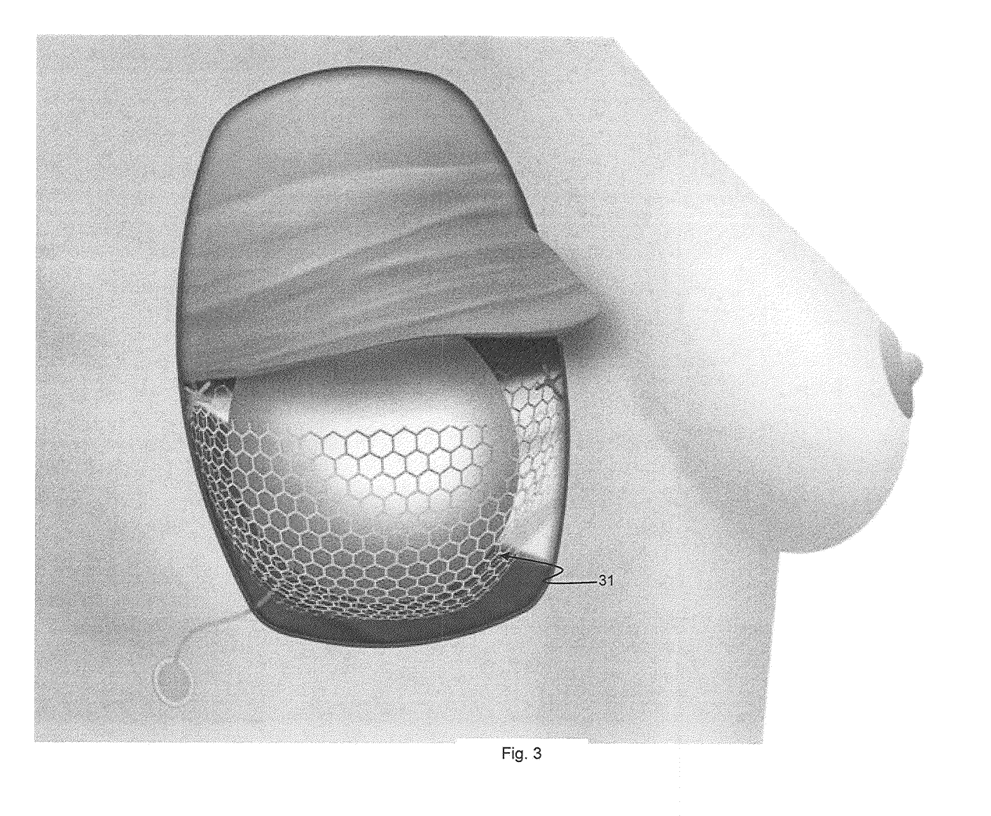

A detailed view of an alternate embodiment of a non-absorbable segment overlaid with absorbable material is show in FIG. 2, which shows a square section of another preferred embodiment where each sectional square is composed of non-absorbable material 21 which is threaded or woven into or superimposed upon a mesh of absorbable material 22. When utilized, the resulting pattern will be areas of the non-absorbable thread 21 adjacent to each other, but separated by an area of tissue which has no direct remaining threads between the non-absorbing sections.

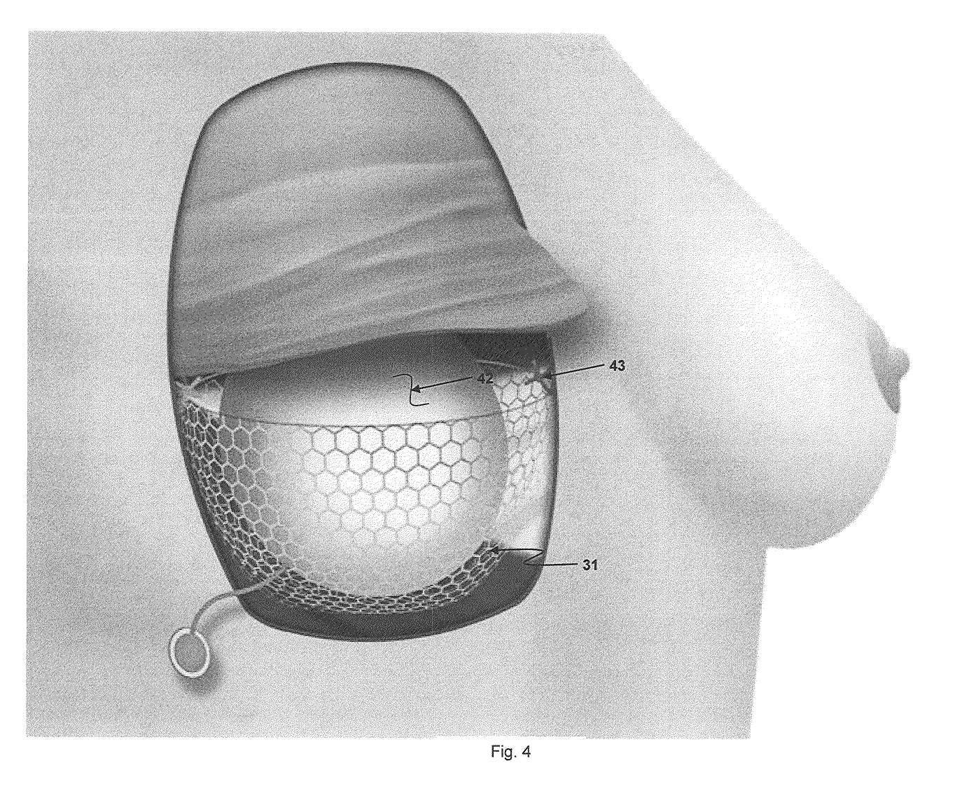

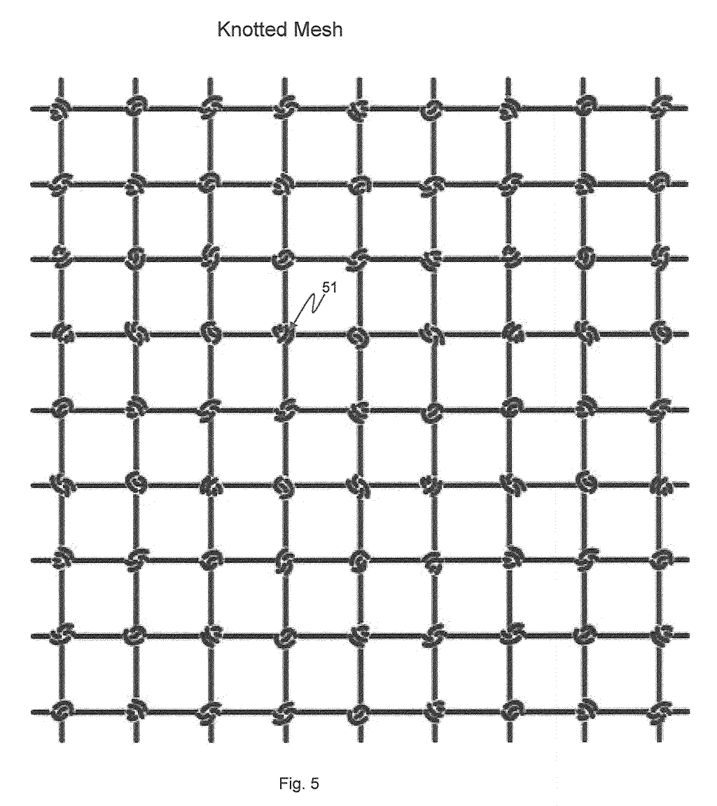

The transitional mesh can be utilized in typical breast implant procedures shown in FIGS. 3 and 4. In FIG. 3 we see the transitional mesh material 31 formed in a cone shape to provide support to the implant. In FIG. 4 we see the transitional mesh material 41 formed into a bag or pocket to contain the implant 42. The sutures 43 confine the movement of the mesh and thereby provide support to the implant during healing, with the non-absorbable components remaining to provide continuing support. Absorbable hooks or transitional thread made up of alternate sections which are absorbable and sections which are non-absorbable can be utilized for sutures as the surgeon desires.

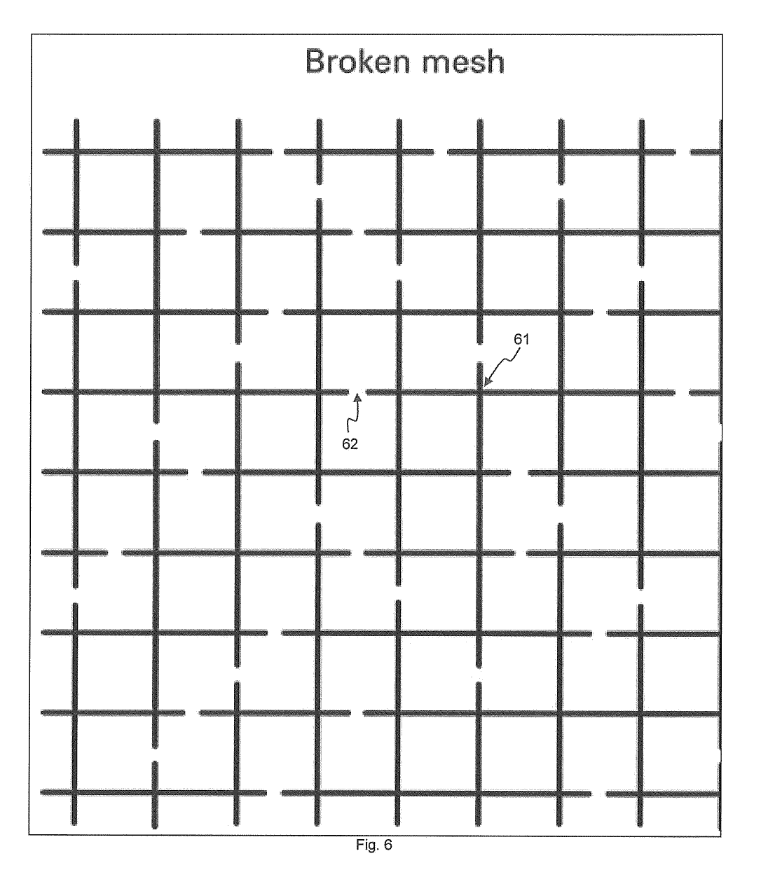

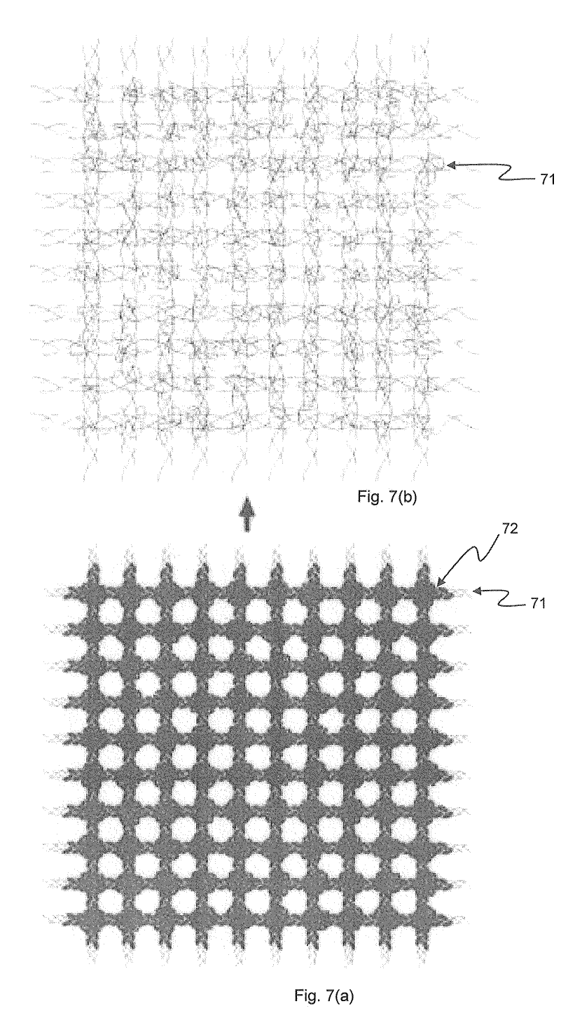

The mesh as shown in FIG. 5 is knotted, wherein each thread in the horizontal and vertical intersection 51 is knotted or fixed with the material of the thread having an inherent elasticity representing a form of yielding mesh. This configuration can be utilized when greater strength is needed. Alternatively, as seen in FIG. 6, where greater movement is desired, a non-knotted thread 61 and/or broken thread 62 can be utilized. This representative of a yielding mesh where yielding is accomplished by the lack of noted connections and/or breaks in the threads. As will be shown the ability to yield and thereby provide a degree of freedom of motion to provide a more natural appearance can also be accomplished using the appropriate weaving of thread or fibers within the thread. Both these fabric type components leave a structure in the body which provides structure and support.

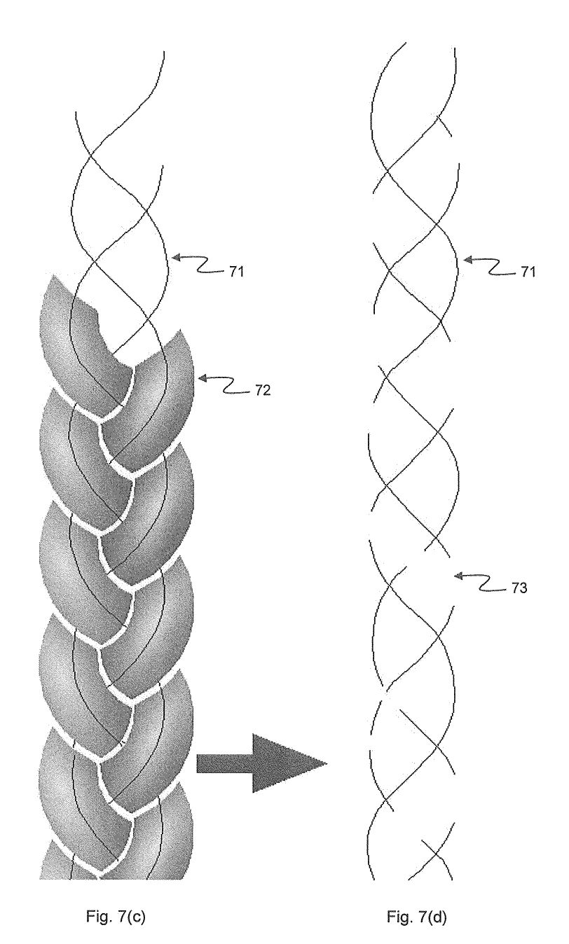

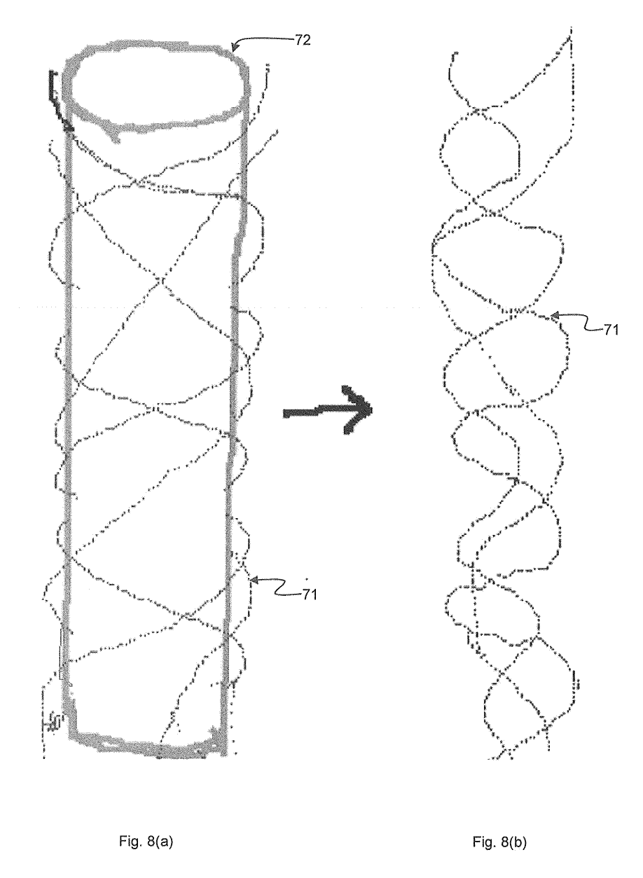

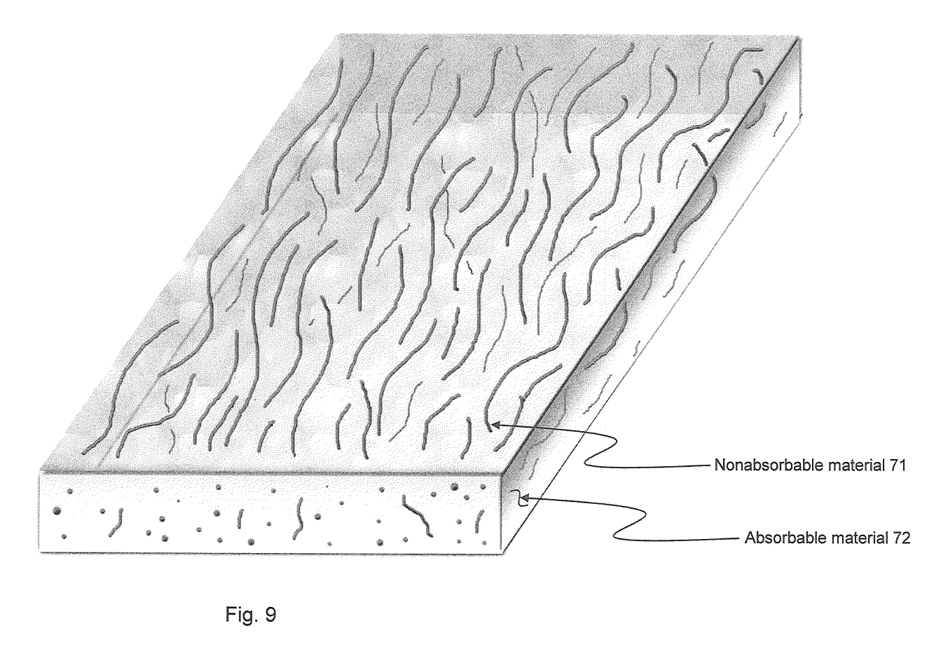

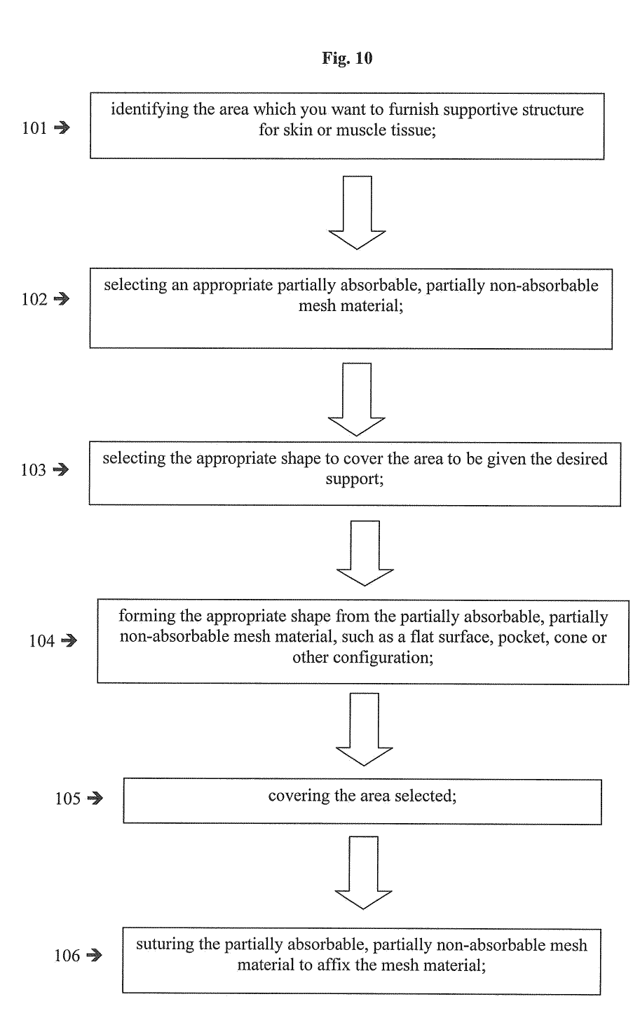

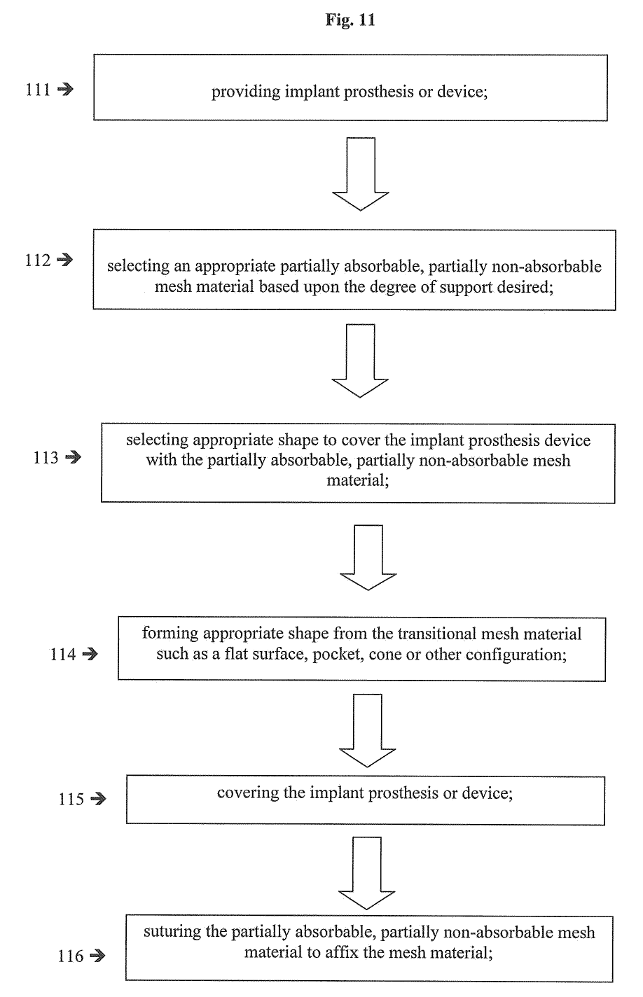

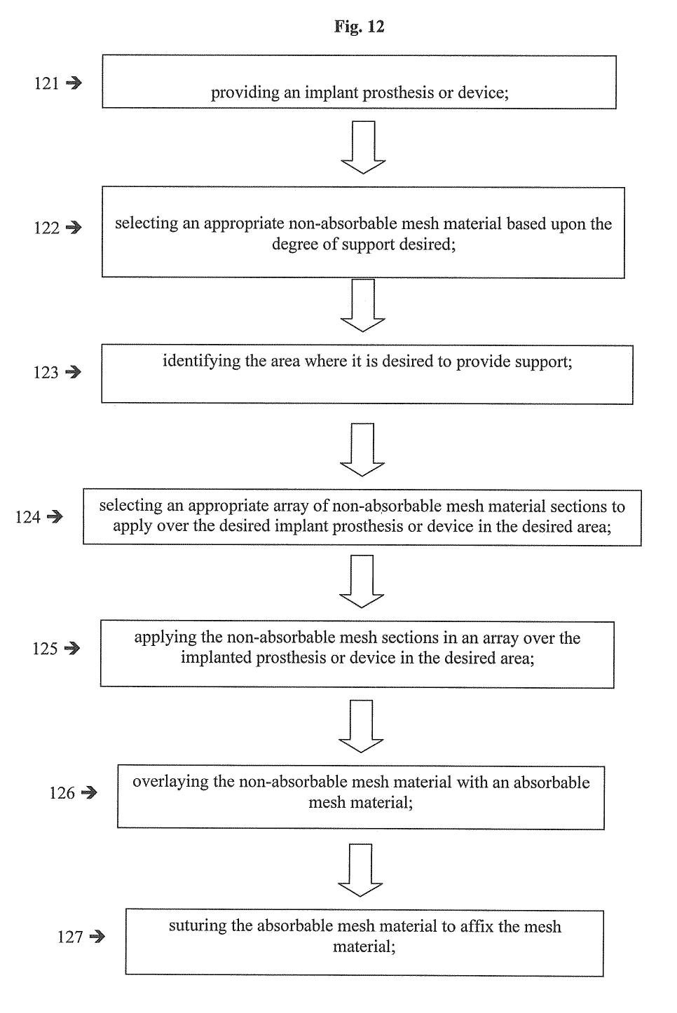

FIG. 7a shows transitional mesh comprising of woven 71 threads housing or covered with an absorbable material 72 which provides temporary support and upon absorption will leave only the woven thread in a woven fabric formation FIG. 7(b). The woven threads provide freedom of movement once the absorbable components 72 are dissipated by virtue of, the loose weaving of the thread components, using an elastic type thread, by using an expandable knitting of the threads or by having the thread components be discontinuous 73 or a combination thereof. The remaining non-absorbable material acts as a scaffold to provide residual strength to the tissue yet allow a degree of freedom of motion. FIG. 7(c) shows the absorbable and non-absorbable transitional thread where the thread non-absorbable material is housed or covered with the absorbable material and the strands of non-absorbable material 71 remain after absorption FIG. 7(d). The elasticity, or ability to elongate, of the thread can be accomplished by the type of weave used for the thread and its components, the thread fibers being discontinuous and having breaks in its continuity 73 either in the thread fiber components or sections of the thread being fully absorbable being followed by an adjacent section being fully non-absorbable, use of an elastic material for the thread component, or any combination of these type or similar constructions. The non-absorbable thread components 71 in FIG. 8(a) can also be wrapped or woven around the absorbable material 72 and in FIG. 8(b) we see the remaining non-absorbable loosely weaved thread 71 after absorption has occurred. The differing types of weaving or threading, size and type of thread, being monofilament or not, and patterns utilized gives one skilled in the art flexibility to select the type of transitional mesh or thread to meet the specific requirements needed.

The area between the non-absorbable segments can contain threads which have non-absorbable fiber components if additional support is desired in the tissue between the non-absorbable segments.

This concept of transitional mesh can be applied to mesh of all types of patterns and sizes to allow its utilization for large areas where a greater portion of the area is composed of the non-absorbable material to smallest areas where the material is reduced to a transitional thread which is composed of a series of non-absorbable sections separated by absorbable sections. The thread when utilized results in discrete, non-absorbable sections remaining in the body to provide support. This transitional thread can be utilized for sutures and can be utilized to create an area where partial support is desired between non-absorbable mesh sections as well as be used in areas which are supported by the thread stitching created by the physician during surgery.

The shape shown of the non-absorbable mesh segments in the preferred embodiment in FIG. 1 and FIG. 2 is hexagonal, but that geometric shape is not required to obtain the benefits desired. Depending on the area, rectangular sections, square sections, random fibers and various other patterns or threads sutured by the physician can also be employed to achieve the desired result of the invention.

It would be desirable but not required that the areas where non-absorbable material is utilized be staggered, or not on continuous straight lines or curves, as more natural motion can be obtained, and discontinuous non-absorbable material reduces formation of scar tissue.

The fabric material in all of the embodiments may also be impregnated or coated with infection-fighting antibiotics or drugs to provide pain relief, to facilitate reduction of scar tissue, or to promote tissue in-growth.

Although the configurations in FIGS. 1 and 2 show the structure of the transitional mesh in two dimensions, that is not a required constraint. The mesh used for the absorbable and non-absorbable segments can have a varied height of surface which promotes tissue in-growth with the segment and helps impede scar tissue formation. Further, the segment or segments of non-absorbable fabric can be varied in size from small to large sections to accomplish the desired support needed.

Although use of the invention has been primarily shown for breast implant utilization, the transitional mesh fabric can be used for other surgical repairs or utilized in areas where additional strength is needed but natural motion and minimal scar tissue formation is desired.

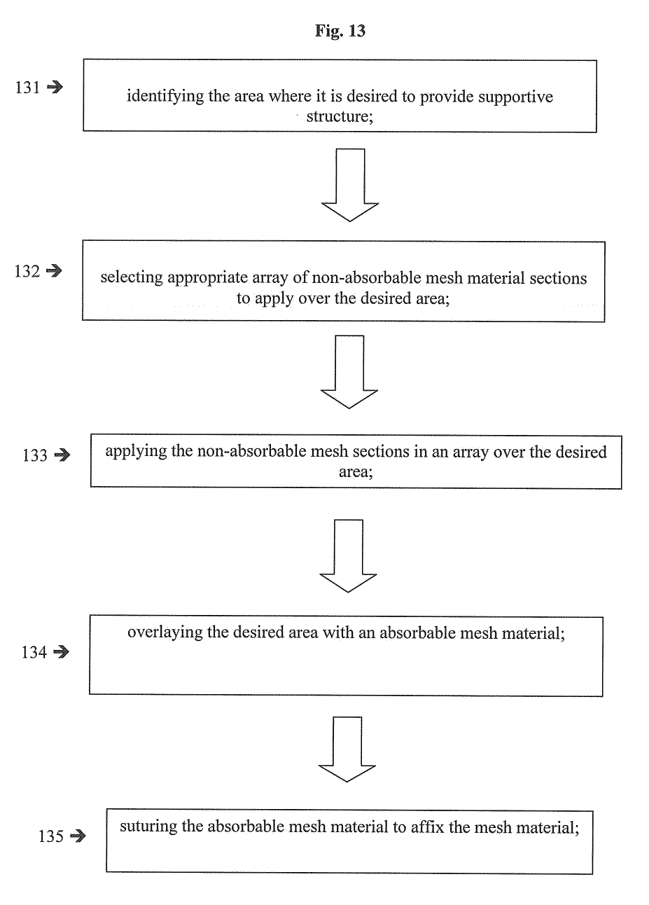

The types of mesh that can be utilized can vary depending upon the needs and desires of the surgeon. The material utilized for the mesh in the preferred embodiments are composed of synthetic material, parts bio-absorbable and parts non-bioabsorable. When used in this patent, the term “absorbable” incorporates “bio-absorbable” and “bio-degradable” and means the composition of the material is broken down or assimilated by the human body and there is no stiffness nor significant tensile strength remaining in the material. The final disposition of the material may or may not result in it being ultimately flushed from the body's tissue. The transitional mesh material may also be composed of threads which themselves are partially absorbable and lose tensile strength. Other mesh materials available include those that are totally biological in nature, e.g. collagen, skin tissue, or a combination of biological and synthetic materials. Variations in type of mesh and material also exist within the mesh structure where some thread materials are knotted, some without knots and the strands are threaded, braided or woven. Another preferred embodiment FIG. 9 would have the absorbable material in a soft sponge-like consistency 72 being formed as a sheet which has intermixed within the absorbable material 72 threads of non-absorbable material 71 which will provide support as the absorbable material dissipates and new tissue forms about the remaining non-absorbable threads. Although in FIG. 9 the non-absorbable threads are shown to run in the direction of one axis, they can be running in any direction depending upon the effect you wish to obtain. Permanent materials can be, but are not limited to, nylon, prolene, polyester, polytetra flourethylene (PTFE), silk, and biological collagen. Absorbable materials may be, but are not limited to cat gut, polyglysolic acid, trimethylene carbonate, and silk. The types of materials used for both non-absorbable material and absorbable material is constantly changing and would be known to one skilled in the art.

Included herein is a set of flow charts representative of exemplary methodologies for performing novel aspects of the disclosed architecture. While, for purposes of simplicity of explanation, the one or more methodologies shown herein, for example, in the form of a flow chart or flow diagram, are shown and described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology could alternatively be represented as a series of interrelated states or events, such as in a state diagram. Moreover, not all acts illustrated in a methodology may be required for a novel implementation.

The invention also contemplates a method for providing support for a breast implant or other prosthesis in a patient. The method includes the steps of providing an implant prosthesis or device then selecting the appropriate transitional mesh material based upon the needed support, selecting the appropriate shape to utilize, forming the transitional mesh to cover the implant prosthesis or device, and then suturing the transitional mesh to provide support. This method may also be utilized for other areas of the body where support is needed for the skin or muscle tissue and no implant prosthesis or device is going to be utilized. The method may also be utilized by constructing the combination of non-absorbing and absorbing mesh sections by cutting patches or segments of non-absorbing mesh and suturing them into patterns with tissue spaces between non-absorbing mesh sections and using non-absorbing or absorbing sutures, fabric sheets or gelatinous material with or without an overlay of absorbing mesh, to provide support to the patterned array of non-absorbing mesh sections.

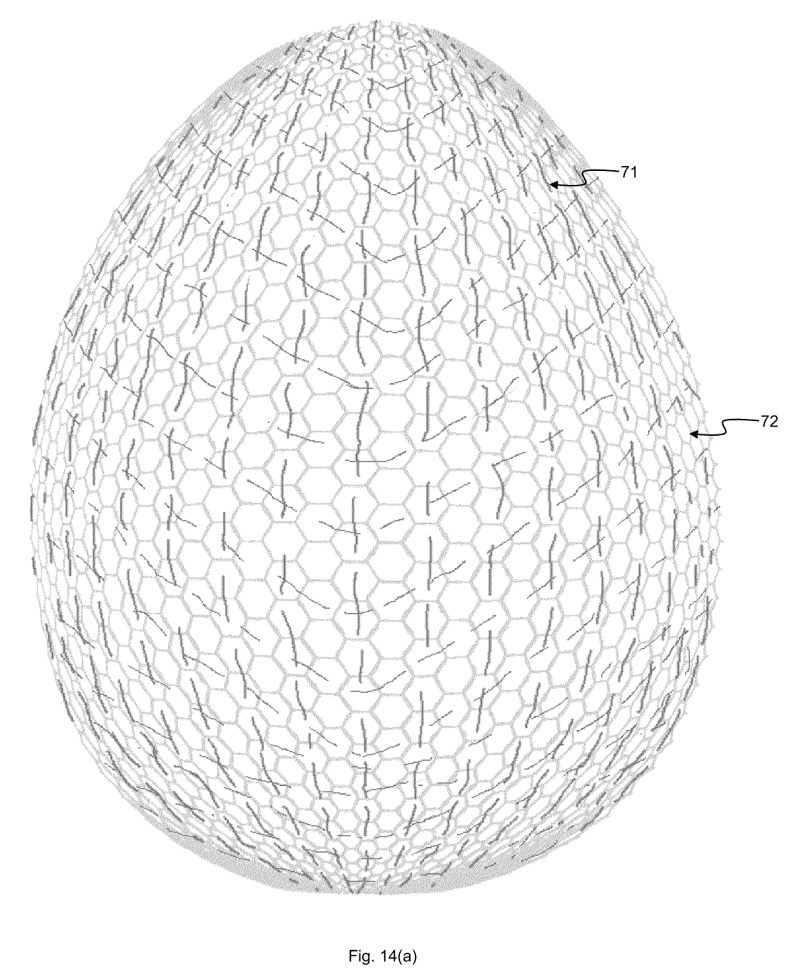

As illustrated in FIG. 10, a method in accordance with the present invention for repair or support of tissue areas comprises the following acts:

first 101 identifying the area which you want to furnish supportive structure for skin or muscle tissue, then

second 102 selecting an appropriate partially absorbable, partially non-absorbable mesh material, then

third 103 selecting the appropriate shape to cover the area to be given the desired support, then

fourth 104 forming the appropriate shape from the partially absorbable, partially non-absorbable mesh material, such as a flat surface, pocket, cone or other configuration, then

fifth 105 covering the area selected

sixth 106 suturing the partially absorbable, partially non-absorbable mesh material to affix the mesh material.

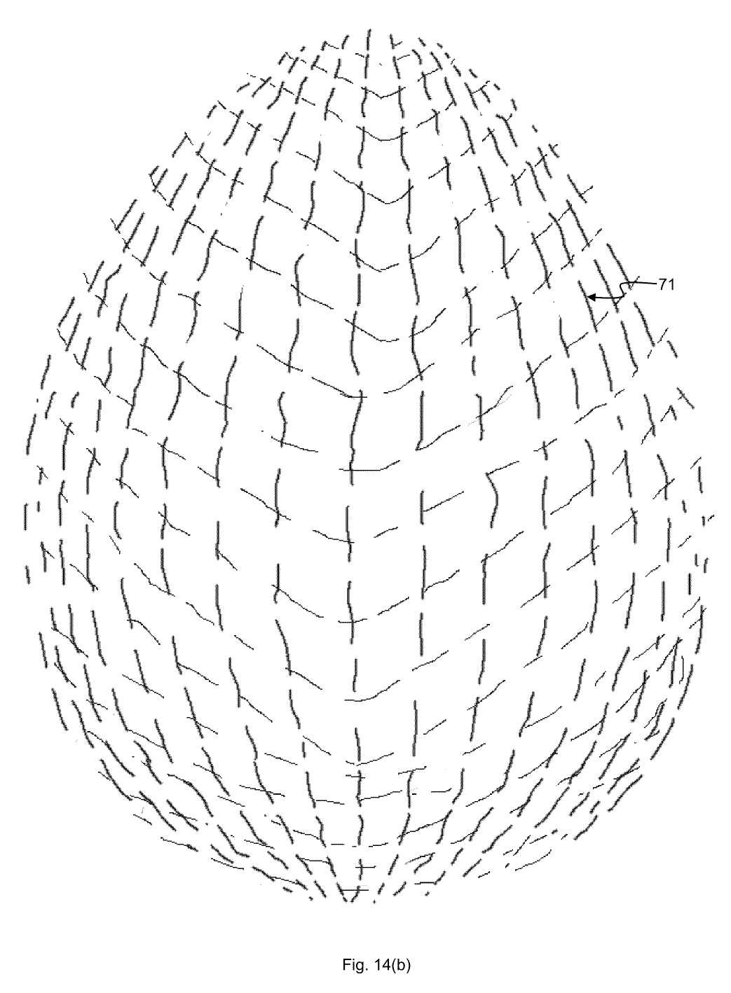

A second method as illustrated in FIG. 11 in accordance with the present invention would be for providing support to an area of the body comprising the following acts:

first 111 providing implant prosthesis or device, then

second 112 selecting an appropriate partially absorbable, partially non-absorbable mesh material based upon the degree of support desired, then

third 113 selecting appropriate shape to cover the implant prosthesis device with the partially absorbable, partially non-absorbable mesh material, then

fourth 114 forming appropriate shape from the transitional mesh material such as a flat surface, pocket, cone or other configuration, then

fifth 115 covering the implant prosthesis or device, then

sixth 116 suturing the partially absorbable, partially non-absorbable mesh material to affix the mesh material.

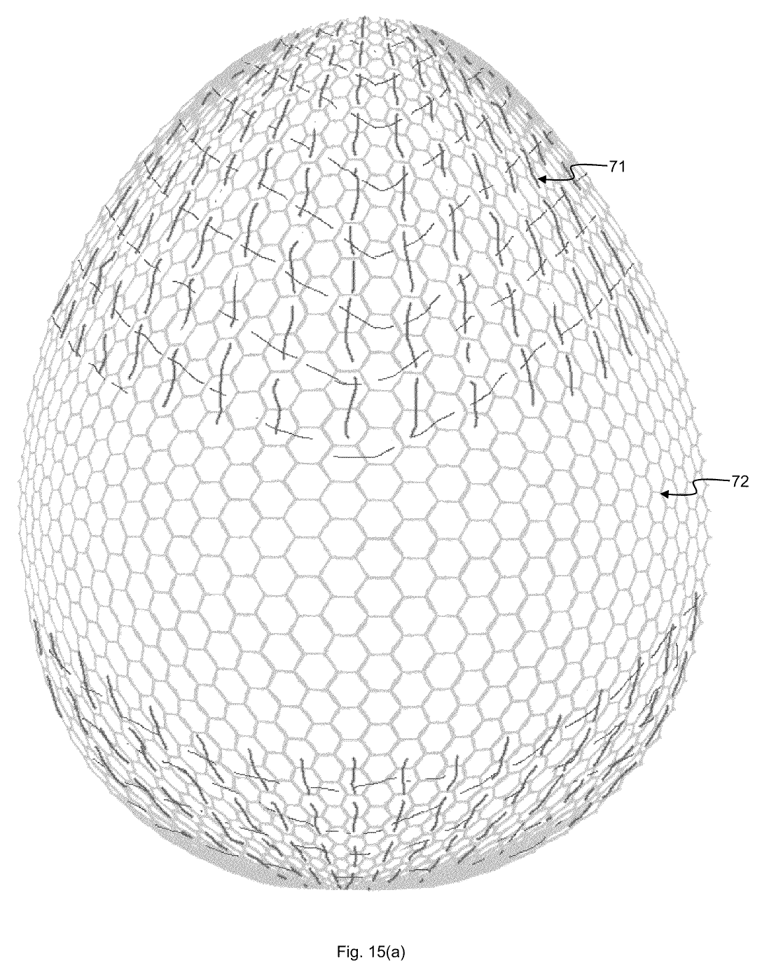

A third method as illustrated in FIG. 12 in accordance with the present invention would be for providing support for a prosthetic device or implant, or to an area of the body by the following acts:

first 121 providing an implant prosthesis or device, then,

second 122 selecting an appropriate non-absorbable mesh material based upon the degree of support desired, then

third 123 identifying the area where it is desired to provide support, then

fourth 124 selecting an appropriate array of non-absorbable mesh material sections to apply over the desired implant prosthesis or device in the desired area, then

fifth 125 applying the non-absorbable mesh sections in an array over the implanted prosthesis or device in the desired area, then

sixth 126 overlaying the non-absorbable mesh material with an absorbable mesh material, then

seventh 127 suturing the absorbable mesh material to affix the mesh material.

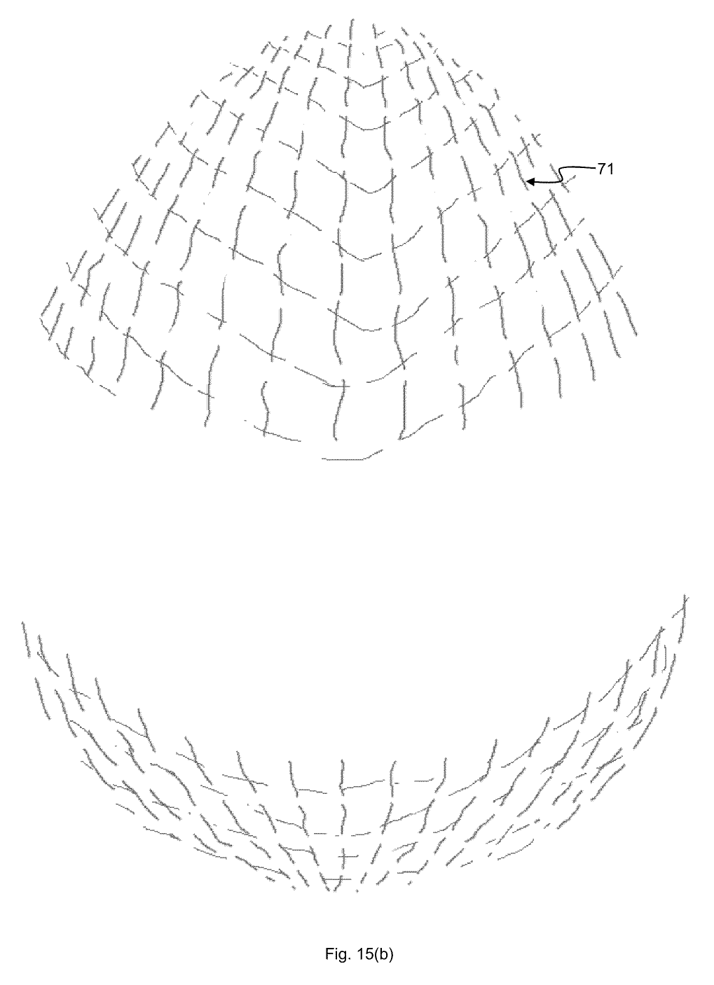

A fourth method as illustrated in FIG. 13 in accordance with the present invention would be for providing support by constructing an array of non-absorbable mesh for repair or support of tissue areas, said method comprising the acts of:

first 131 identifying the area where it is desired to provide supportive structure, then

second 132 selecting appropriate array of non-absorbable mesh material sections to apply over the desired area, then

third 133 applying the non-absorbable mesh sections in an array over the desired area, then

fourth 134 overlaying the desired area with an absorbable mesh material, then

fifth 135 suturing the absorbable mesh material to affix the mesh material.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed:

1. A biocompatible meshed material for implanting into the body to provide support comprising:
a plurality of non-absorbable mesh sections, placed into an array over a desired area,
wherein the sections are not all contiguously placed over the desired area; and
absorbable mesh material,
wherein the non-absorbable mesh sections are overlaid with the absorbable mesh material.

2. The meshed material of claim 1,
wherein the non-absorbable mesh sections are comprised of loosely woven fibers.

3. The meshed material of claim 1,
wherein the non-absorbable mesh sections are coated with the absorbable mesh material.

4. The meshed material of claim 2,
wherein the loosely woven fibers of the non-absorbable mesh sections are broken and not knotted.

5. The meshed material of claim 1,
wherein the non-absorbable mesh sections comprise threads that are knotted.

6. The meshed material of claim 1,
wherein the non-absorbable mesh sections are coated with an antibiotic material.

7. The meshed material of claim 1,
wherein the non-absorbable mesh sections are coated with material to reduce scar tissue formation.

8. The meshed material of claim 1,
wherein the non-absorbable mesh sections are coated with material to promote tissue growth.

9. The meshed material of claim 1,
wherein the non-absorbable mesh sections are coated with a medicinal material.

10. The meshed material of claim 5,
wherein the threads of the non-absorbable mesh sections are elastic.

11. A biocompatible fabric of meshed material for implanting into the body to provide support comprising:
a woven fabric in the form of a mesh,
a plurality of sections of the mesh comprising non-absorbable fibers,
wherein the non-absorbable fibers are placed in an array; and
portions of space between adjacent sections of the mesh comprising absorbable material.

12. The fabric of claim 11,
wherein the non-absorbable fibers are surrounded by absorbable material at perimeter edges.

13. The fabric of claim 11,
wherein the woven fabric is configured in various thicknesses.

14. The fabric of claim 11,
wherein the woven fabric is coated with the absorbable material.

15. The fabric of claim 11,
wherein the woven fabric is coated with an antibiotic material.

16. The fabric of claim 11,
wherein the woven fabric is coated with material to reduce scar tissue formation.

17. The fabric of claim 11,
wherein the woven fabric is coated with material to promote tissue growth.

18. The fabric of claim 11, wherein the woven fabric is coated with a medicinal material.

19. A biocompatible suturing material for implanting into the body comprising:
a thread comprising:
a non-absorbing material fiber section; and
an absorbable material fiber section,
wherein the non-absorbable fiber section is loosely woven.

20. The suturing material of claim 19,
wherein the thread is comprised of fibers which are loosely woven around absorbable material.

21. The suturing material of claim 20,
wherein the thread fibers are not continuous.

22. The suturing material of claim 19,
wherein the thread is coated with absorbable material.

23. The suturing material of claim 19,
wherein the thread is coated with an antibiotic material.

24. The suturing material of claim 19,
wherein the thread is coated with a material to reduce scar tissue formation.

25. The suturing material of claim 19,
wherein the thread is coated with material to promote tissue growth.

26. The suturing material of claim 19,
wherein the thread is coated with a medicinal material.

27. A biocompatible sheet material for implanting into a body to provide support comprising:
an absorbable material in the form of a sheet; and
a plurality of non-absorbable threads,
wherein the non-absorbable threads are distributed within the absorbable material.

28. The sheet material of claim 27,
wherein the absorbable sheet material has a varying thickness.

29. The material of claim 27,
wherein the consistency of the absorbable sheet material is sponge-like.

30. A method for providing support for skin or muscle tissue, said method comprising the acts of:
identifying area to furnish supportive structure for skin or muscle tissue;
selecting an appropriate partially absorbable, partially non-absorbable mesh material;
selecting an appropriate shape to cover an area to provide desired support;
forming the appropriate shape from the appropriate partially absorbable, partially non-absorbable mesh material;
covering area selected; and
suturing the appropriate partially absorbable, partially non-absorbable mesh material to affix mesh material.

31. A method of providing support for an implant prosthesis or device, said method comprising the acts of:
providing an implant prosthesis or device;
selecting an appropriate partially absorbable, partially non-absorbable mesh material based upon degree of support desired;
selecting an appropriate shape to cover the implant prosthesis or device with the appropriate partially absorbable, partially non-absorbable mesh material;
forming the appropriate shape from the appropriate partially absorbable, partially non-absorbable mesh material;
covering the implant prosthesis or device; and
suturing the appropriate partially absorbable, partially non-absorbable mesh material to affix mesh material.

32. A method for providing support for an implant prosthesis or device, said method comprising the acts of:
providing an implant prosthesis or device;
selecting an appropriate non-absorbable mesh material based upon degree of support desired;
identifying an area to provide support;
selecting an appropriate array of non-absorbable mesh material sections to apply over the desired implant prosthesis or device in the identified area;
applying the non-absorbable mesh material sections in the selected array over the implant prosthesis or device in the identified area;
overlaying the non-absorbable mesh material sections with an absorbable mesh material; and
suturing the absorbable mesh material to affix mesh material.

33. A method for providing support for skin or muscle tissue, said method comprising the acts of:
identifying area to provide supportive structure;
selecting an appropriate array of non-absorbable mesh material sections to apply over the identified area;
applying the non-absorbable mesh material sections in the selected array over the identified area;
overlaying the identified area with an absorbable mesh material; and
suturing the absorbable mesh material to affix mesh material.
